(19) United States
(12) Patent Application Publication
(10) Pub. No.: US 2017/0007127 A1
(43) Pub. Date: Jan. 12, 2017

(54) Diagnostic Service Providing System and Diagnostic Service Providing Method

(71) Applicant: Hitachi, Ltd., Tokyo (JP)

(72) Inventor: Kei TAKENAKA, Tokyo (JP)

(21) Appl. No.: 15/147,316

(22) Filed: May 5, 2016

(30) Foreign Application Priority Data
Jul. 8, 2015 (JP) 2015-137167

(51) Int. Cl. A61B 5/00; A61B 5/08; G06F 19/00; A61B 5/145

(52) U.S. Cl. CPC A61B 5/0022; A61B 5/145; A61B 5/14507; A61B 5/08; A61B 5/72; G06F 19/3418

(57) ABSTRACT

A diagnostic result is immediately presented using a diagnostic apparatus installed in a diagnostic apparatus installation facility. Provided is a diagnostic service system which includes a diagnostic apparatus installed in a diagnostic apparatus installation facility and performing a diagnosis using a disposable device, a user terminal owned by a user of the diagnostic apparatus, and an information processing apparatus connected with the diagnostic apparatus and the user terminal and gathering a diagnostic result of the diagnostic apparatus, in which the information processing apparatus manages information of the diagnostic result diagnosed by the diagnostic apparatus and information of the user by associating the both with each other, and provides the diagnostic result in accordance with an access from the user terminal of the user.

### TECHNICAL FIELD

The present invention relates to a diagnostic service providing system and a diagnostic service providing method, and is suitably applied to a diagnostic service providing system and a diagnostic service providing method in which a diagnosis is performed with a diagnostic apparatus using a disposable device.

### BACKGROUND ART

The present invention relates to a service method for diagnosing an analyte sample such as blood, urine, or exhalation collected by a user himself/herself with a diagnostic apparatus using a disposable device, and more particularly to a diagnostic service method using electronic commerce.

When it is intended to live a healthy life, it is required to find a disease before onset of the disease or at an initial stage of the disease and receive appropriate treatment in a medical institution. However, even today, where a medical system is established, there is a case where a disease is left without receiving diagnosis in a medical institution and symptoms of a disease become serious, due to the burden of visiting a medical institution or the like.

In recent years, there has been a disease diagnostic method using a simple inspection kit as a method for receiving a diagnosis of a disease without visiting a medical institution. In the diagnostic method, a user purchases the inspection kit, collects an analyte such as blood, urine, or feces using the inspection kit, and then sends the collected analyte to an inspection institution. The user can receive an inspection result via mail or the Internet after several days from the date when the analyte was sent. Such an inspection kit for use at home targets a cancer inspection for colon cancer or the like and a disease, which requires a long period of time for treatment, for example, lifestyle-related diseases such as AIDS, hepatitis C, and diabetes.

For example, PTL 1 discloses a diagnostic service using an inspection kit installed in an inspection kit installation facility. Specifically, the user brings the inspection kit installed in the inspection kit installation facility home and then, collects the analyte such as blood or urine by himself/herself. Then, the user sends the collected inspection kit to the inspection institution directly or through the inspection kit installation facility and thus, obtains a result of an inspection conducted in the inspection institution over the Internet.

### CITATION LIST

### Patent Literature

PTL 1: JP-A-2007-47856

### SUMMARY OF INVENTION

### Technical Problem

The inspection using the simple kit and the Internet described in PTL 1 has an advantage that the user is capable of being inspected without visiting a medical institution or an inspection institution. However, there is a problem that the collected analyte cannot be immediately inspected and the analyte is changed with time, or it takes a long time to receive a diagnostic result in a case where treatment should be made immediately after the onset of a disease.

The present invention has been made in consideration of the problem described above and provides a diagnostic service providing system and a diagnostic service providing method capable of immediately presenting a diagnostic result using a diagnostic apparatus installed in a diagnostic apparatus installation facility.

### Solution to Problem

In the present invention for solving the problem described above, there is provided a diagnostic service system which includes a diagnostic apparatus installed in a diagnostic apparatus installation facility and performing a diagnosis using a disposable device, a user terminal owned by a user of the diagnostic apparatus, an information processing apparatus connected with the diagnostic apparatus and the user terminal and gathering a diagnostic result of the diagnostic apparatus, in which the information processing apparatus manages information of the diagnostic result diagnosed by the diagnostic apparatus and information of the user by associating the both with each other and provides the diagnostic result in accordance with an access from the user terminal of the user.

In the present invention for solving the problem described above, there is provided a diagnostic service providing method in a diagnostic service system including a diagnostic apparatus installed in a diagnostic apparatus installation facility, a user terminal owned by a user of the diagnostic apparatus, an information processing apparatus connected with the diagnostic apparatus and the user terminal and gathering a diagnostic result of the diagnostic apparatus, and the diagnostic service providing method includes a step of, by the diagnostic apparatus, sending the diagnostic result and contact information of the user to the information processing apparatus of an information manager, a step of, by the information management apparatus, sending an address in which information of the diagnostic result is written, a step of, by the user terminal, accessing the address in which the diagnostic result is written in accordance with an input by the user to obtain information of the diagnostic result, a lot number of the diagnostic result, and the medical institution, a step of providing the lot number of the diagnostic result to an information processing terminal of the medical institution through the user terminal in a case where the user has consultation in the medical institution, a step of, by the information processing terminal of the medical institution, sending the lot number of the diagnostic result to the information processing apparatus to acquire the information of the diagnostic result, and a step of, by the information processing terminal of the medical institution, sending a diagnostic result of the medical institution to the information processing apparatus.

### Advantageous Effects of Invention

According to the present invention, it is possible to immediately present a diagnostic result using a diagnostic apparatus installed in a diagnostic apparatus installation facility and find a disease of a user at an early stage without visiting a medical institution.

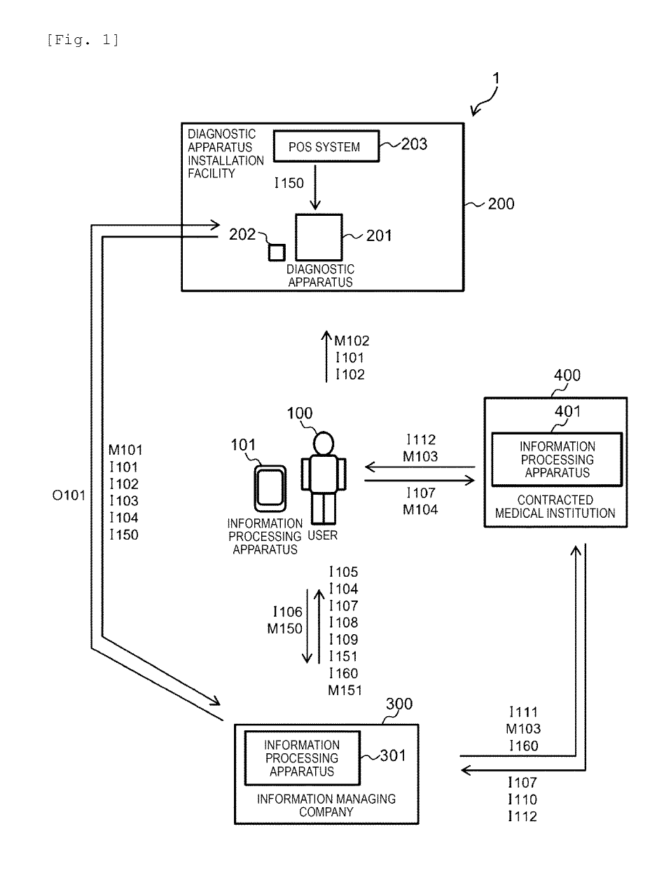

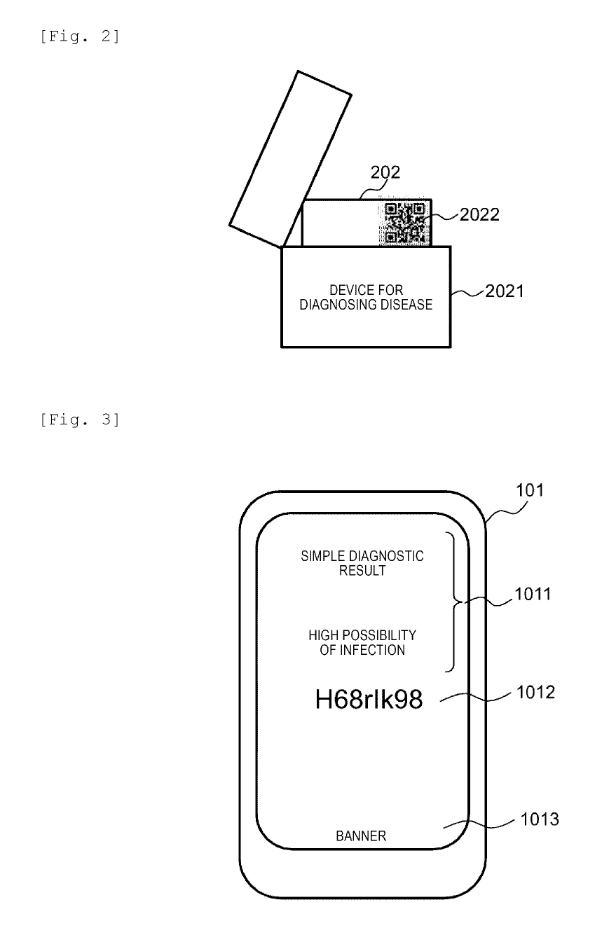

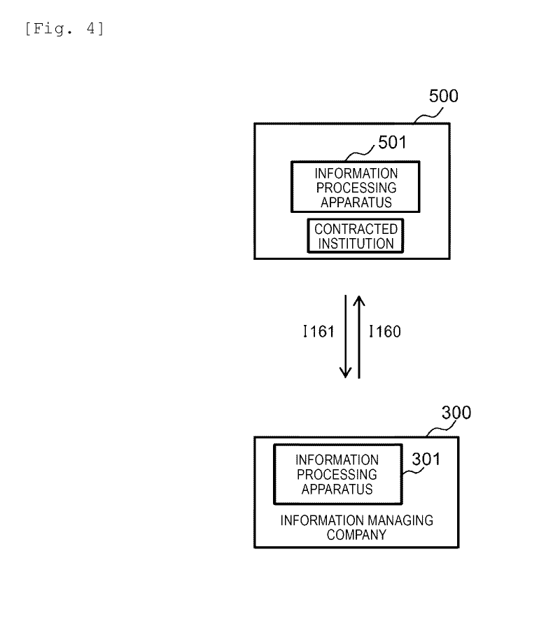

### BRIEF DESCRIPTION OF DRAWINGS

[FIG. 1] FIG. 1 is a diagram conceptually illustrating a configuration of a diagnostic service system according to an embodiment of the invention.

[FIG. 2] FIG. 2 is a diagram conceptually illustrating a configuration of a disposable device according to the embodiment.

[FIG. 3] FIG. 3 is a diagram conceptually illustrating an example of display of a simple diagnostic result according to the embodiment.

[FIG. 4] FIG. 4 is a diagram conceptually illustrating a flow of information between an information managing company and a contracted institution according to the embodiment.

### DESCRIPTION OF EMBODIMENTS

As described above, for the prevention of a disease from becoming serious, it is important to find a disease before the onset of the disease or at an initial stage and receive appropriate treatment in a medical institution. To do this, it is preferable that a user simply conducts a diagnosis by himself/herself even without visiting a medical institution. The inventors of the invention have studied various methods and systems for self-diagnosis and reached the invention.

In the following drawings, an embodiment of the present invention will be described in detail. In the meantime, the embodiment which will be described later is an example, and it is needless to say that other aspects by a combination of respective embodiments with each other, a combination or replacement with well-known art or frequently used art may be made.

(1) Summary of the Present Embodiment

As described above, the inspection using the simple kit and the Internet has an advantage that the user is capable of conducting the inspection without visiting the medical institution or the inspection institution. However, since it takes time for sending a simple kit which has collected the analyte to the inspection institution, it is difficult to immediately inspect the collected analyte and is unsuitable for inspection of substance which is contained in the analyte and easily changed with time. Further, since it is a method having difficulty in acquiring the diagnostic result immediately after the analyte is collected, it is an inspection method unsuitable for a disease which requires treatment immediately after the onset of a disease such as infectious diseases of the respiratory system such as influenza.

Furthermore, when the user who acquires the inspection result intends to receive a diagnosis and treatment of the medical institution, the user needs to look for a medical institution suitable for symptoms of the disease, and there is also a case where the necessity of receiving the same inspection again is caused in a case where the user has consultation in the medical institution which does not use a result of an inspection by the simple kit as a diagnostic material. When the user presents the inspection result of the simple kit to the medical institution, it is necessary to prevent the inspection result from being leaked to a third person.

In the present embodiment, the user conducts a simple diagnosis by himself/herself by the diagnostic apparatus installed in the diagnostic apparatus installation facility and thus, it is possible to immediately present the diagnostic result to the user and select an optimum medical institution for treatment. With this, the user can receive the simplified diagnostic result by the diagnostic apparatus immediately over the Internet and can find the disease of the user at an early stage without visiting the medical institution.

(2) Configuration of Diagnostic Service System

Next, a configuration of a diagnostic service system 1 according to the present embodiment will be described with reference to FIG. 1. As illustrated in FIG. 1, the diagnostic service system 1 is constituted with an information processing apparatus 101 which is able to access the Internet, a diagnostic apparatus 201, an information processing apparatus 301 for simple diagnosis which receives information from the diagnostic apparatus 201 using a disposable device 202 and stores the information, and an information processing apparatus 401 for a medical institution which saves diagnostic records by the medical institution, and these apparatuses are connected to each other over the Internet.

The information processing apparatus 101 (user terminal of the invention) is owned by a user 100 who wants to make a simple diagnosis. The diagnostic apparatus 201 is installed in a diagnostic apparatus installation facility 200. The information processing apparatus 301 is owned by an information managing company 300 which is an operator and an administrator of the diagnostic service system 1. The information processing apparatus 401 for a medical institution is owned by a contracted medical institution 400 which has concluded a contract with the information managing company 300.

As illustrated in FIG. 1, in order to provide the user 100 with a diagnostic service, the information managing company 300 lends out the diagnostic apparatus 201 for a fee or free to the diagnostic apparatus installation facility 200 and supplies the disposable device 202 for a fee or free (O101). In a case where the diagnostic apparatus 201 is lent out for a fee or the disposable device 202 is a device for a fee, the administrator of the diagnostic apparatus installation facility 200 pays a lending fee or a fee of an article of consumption of the disposable device 202 to the information managing company 300 (M101).

The diagnostic apparatus installation facility 200 is a retail store such as a drug store or a convenience store, a staying facility such as a hotel, or an educational facility such as a school or a day-care center. The information managing company 300 is a company, which handles a large quantity of customer information, such as an insurance company.

The disposable device 202 is a disposable device having a function necessary for detecting a specific substance related to a specific case from blood, urine, exhalation, or the like of the user 100. The diagnostic apparatus 201 automates a detection process performed in the disposable device 202 and specifies an amount of the specific substance detected by the disposable device 202. Information about the specific substance is transmitted from the diagnostic apparatus 201 to the information processing apparatus 301 via the network. The information processing apparatus 301 diagnoses the presence or absence or a degree of progress of the disease of the user 100 on the basis of the transmitted information.

(3) Procedural Sequence of Diagnostic Service

Next, a procedural sequence of a diagnostic service will be described.

(3-1) Simple Diagnostic Processing

The user 100 who wants to make a simple diagnosis visits the diagnostic apparatus installation facility 200, pays a fee for an apparatus diagnosis (M102), and purchases the disposable device 202.

The disposable device 202 is essential for the diagnosis of the diagnostic apparatus 201. Therefore, the diagnostic apparatus 201 maybe allowed to be used even without a usage ticket or a usage card for using the diagnostic apparatus 201. The user 100 collects the analyte such as the blood, urine, or exhalation into the disposable device 202 by himself/herself and sets the disposable device 202 which has collected the analyte in the diagnostic apparatus 201.

After the disposable device 202 is set, an email address (I101) and a password (I102) of the user 100 are transmitted to the information processing apparatus 301 through the diagnostic apparatus 201.

The diagnostic apparatus 201 which has received the email address and the password of the user reads a lot number 2012 of the disposable device 202 and selects an optimum process from a plurality of detection processes recorded previously. Thereafter, the diagnostic apparatus 201 automatically conducts the selected detection process and detects the specific substance contained in the collected analyte.

The information about a kind or an amount of the detected specific substance (I104) and the lot number 2022 (I103) of the disposable device 202 are transmitted to the information processing apparatus 301 through a network. The information processing apparatus 301 diagnoses the symptoms of the user 100 on the basis of the information about the kind or the amount of the detected specific substance sent.

(3-2) Provision of Simple Diagnostic Result

The information processing apparatus 301 writes the obtained simple diagnostic result into an electronic medical chart of the user 100 prepared in the information processing apparatus 301 in advance. In this case, personal information such as an age of the user 100 and information such as a place of a diagnostic apparatus installation facility 200 or a diagnosis date in addition to the simple diagnostic result are written into the electronic medical chart.

After the completion of writing into the information processing apparatus 301, the information processing apparatus 301 sends information of an address in which the simple diagnostic result is described to the user 100 (I105). Since the address in which the simple diagnostic result is described is prepared from the lot number 2022 of the disposable device 202 and the email address of the user 100, even though a third person obtains the lot number 2022 of the disposable device 202 or the email address of the user 100, it is impossible to obtain the address in which the simple diagnostic result is described.

Further, in a case where the user 100 uses the diagnostic service system 1 for the first time, the information processing apparatus 301 prepares an electronic medical chart having the email address input by the user 100 as an ID number. Since the electronic medical chart is not prepared in the diagnostic apparatus 201 but in the information processing apparatus 301, even in a case where the user 100 visits another diagnostic apparatus installation facility 200 and uses the diagnostic apparatus 201 of the diagnostic apparatus installation facility 200, the user 100 inputs the email address and the password and thus, another simple diagnostic result is written into the electronic medical chart of the user 100.

Further, only the newest diagnostic result of information recorded in the electronic medical chart may be provided to the user 100. With this, even in a case where the user 100 visits a plurality of medical institutions, it is possible to always input and output the newest diagnostic result.

(3-3) Provision of Contracted Medical Institution Information or the Like

The user 100 accesses the address sent from the information processing apparatus 301 through the information processing apparatus 101, inputs the password set in advance, and obtains the simple diagnostic result (I104) and the lot number of diagnostic result (I107). Information obtained by the user 100 is only the simple diagnostic result and the lot number of diagnostic result among the information described in the electronic medical chart.

The user 100 can receive information (I108) of the contracted medical institution 400 which has concluded a contract with the information managing company 300 and information of the article that the diagnostic apparatus installation facility 200 handles as an advertisement in accordance with the result of a simple diagnostic result (I109).

Selection of the article handled by the contracted medical institution 400 or the diagnostic apparatus installation facility 200 is performed by the information processing apparatus 301 using contents of the simple diagnostic result, personal information such as an address or age of the user 100, or a business state of the contracted medical institution 400 as the determination basis. The individualized advertisement is provided to the user 100 and thus, it is possible to enhance interest in the diagnosis in the contracted medical institution 400 or the article handled by the diagnostic apparatus installation facility 200.

(3-4) Sending of Information to Contracted Medical Institution

In a case where the above-described simple diagnostic result is positive, the information of the contracted medical institution 400 is sent to the user 100. The user 100 visits the contracted medical institution 400 and receives a diagnosis and treatment from the contracted medical institution 400 and thus, it is possible to prevent deterioration of a user due to disease symptoms of a disease at an early stage.

The contracted medical institution 400 receives the password (I110) which was input to the information processing apparatus 301 at the time of concluding the contract with the information managing company 300. The contracted medical institution 400 can receive the lot number of the simple diagnostic result (I107) from the user 100 at the time of clinical consultation and input the password (I110) so as to obtain the simple diagnostic result from the electronic medical chart of the user 100 within the information processing apparatus 301.

In this case, the contracted medical institution 400 can also obtain information (I111) such as a place of the diagnostic apparatus installation facility 200, a diagnosis time, and a past diagnostic history in addition to the diagnostic result obtained by the user 100. The contracted medical institution 400 can predict a period and a place of onset of the disease for the user 100 from the diagnostic result for the medical institution (I111).

(3-5) Diagnosis of Contracted Medical Institution

The user 100 can pay a consultation fee to the contracted medical institution 400 so as to receive the diagnosis from the contracted medical institution 400. Since the diagnosis of symptoms becomes easy by obtaining the information of the simple diagnostic result, the contracted medical institution 400 can reduce the time and labor required for clinical consultation of a user. Then, the user 100 may be allowed to receive a discount on the consultation fee by presenting the lot number of the simple diagnostic result to the contracted medical institution 400.

The contracted medical institution 400 may receive an amount of discount for the clinical consultation from the information managing company 300. Specifically, the contracted medical institution 400 pays the amount of discount for the clinical consultation fee (M103) to the user 100 who has performed payment of the consultation fee M104. Thereafter, the contracted medical institution 400 transmits a clinical consultation result of the medical institution I112 and the lot number of diagnostic result I107 to the information managing company 300 and receives the amount of discount for the clinical consultation fee (M103) corresponding to the amount paid to the user 100 from the information managing company 300.

As described above, when the diagnostic result by the contracted medical institution 400 is transmitted to the information processing apparatus 301, the simple diagnostic result for the disease of the user 100 and the diagnostic result by the contracted medical institution are gathered in the information processing apparatus 301. Performance of the diagnostic apparatus 201 can be evaluated by collating the simple diagnostic result with the diagnostic result of the contracted medical institution and the performance of the diagnostic apparatus 201 can be improved on the basis of an evaluation result.

(3-6) Provision of Insurance Product

In a case where a POS system (point-of-sales information management diagnosis) 203 is provided in the diagnostic apparatus installation facility 200, the personal information such as the age or sex of the user 100 (I150) and the simple diagnostic result may be provided to the information managing company 300 by making the diagnostic apparatus 201 cooperate with the POS system 203. With this, the information managing company 300 can estimate an age group or sex with a lot of patients.

In a case where the information managing company 300 has a function as an insurance company, it is possible to provide the user 100 with individualized information of the insurance product (I151) on the basis of the above-described personal information (I150). Since the user 100 who conducts the self-diagnosis using the diagnostic apparatus 201 can be determined as a customer who is highly conscious about his/her health by the information managing company 300, the insurance product for which an insurance fee is discounted can be provided with lower risk (M150, M151).

(3-7) Provision of Health and Infection Information

Information of the simple diagnostic results aggregated from the diagnostic apparatus 201 installed in a plurality of the diagnostic apparatus installation facilities 200 is gathered in the information processing apparatus 301 of the information managing company 300. The information of the simple diagnostic results is arranged for each group of an area, age, sex, or the like and thus, an occurrence state of a disease can be clarified in each group.

Information about the occurrence state of the disease can be appropriately transmitted to the user 100 or the contracted medical institution 400 by the information managing company 300 and the user 100 can take appropriate preventive measures from the transmitted information. The user 100 receives the health and infection information I160 from the information managing company 300 can take measures such as precautions in advance. The contracted medical institution 400 receives the health and infection information I160 from the information managing company 300 and can predict the number or medical examination item of the user 100 who receives the clinical consultation of the contracted medical institution 400 from the transmitted information and arrange a consultation system in advance.

Next, descriptions will be made on a disposable device 202 with reference to FIG. 2. FIG. 2 illustrates the disposable device 202 immediately after being opened. As illustrated in FIG. 2, the disposable device 202 is stored in a box 2021 in which a diagnostic disease name is described, the box 2021 is opened immediately before being used, and the disposable device 202 is taken out.

In order to protect diagnostic performance of the disposable device 202, it is preferable that the box 2021 is sealed or has light shielding performance. The lot number 2022 is printed in the disposable device 202. The diagnostic apparatus 201 is caused to read the lot number 2022 and thus, the diagnostic apparatus 201 can automatically conduct a diagnostic process corresponding to each disease. With this, it is possible to prevent a failure occurring due to erroneous selection of the diagnostic process by the user 100.

FIG. 3 is an example of display of a simple diagnostic result 1011 displayed on the information processing apparatus 101 by accessing the information processing apparatus 301 through the information processing apparatus 101 by the user 100. A lot number 1012 for identifying the diagnostic result is allocated to the diagnostic result of the simple diagnostic result 1011. The lot number 1012 is associated with the lot number 2022 of the disposable device 202, an installed place or a use time of the diagnostic apparatus 201, and information of the user 100, and can check the information as necessary.

It is possible to display a banner 1013 and a simple diagnostic result 1012. As described above, it is possible to change the contents such as information of the contracted medical institution 400 or an advertisement to be displayed in the banner 1013 according to the contents of the diagnostic result of the simple diagnostic result 1011.

For example, in a case where it is expected that a possibility of infection is high and a disease will occur in the near future, information of the contracted medical institution 400 is displayed on the banner. With this, it is possible to urge the user 100 to receive the diagnosis and treatment at an early stage.

Further, it is possible to promote purchase intention by causing a cold medicine or energy drink handled by the diagnostic apparatus installation facility 200 to be displayed. When the diagnostic result is determined as negative, it may be allowed to display the advertisement of a product or service having a high possibility of being preferred by the user 100 on the basis of the information of the POS system 203 without displaying the information described above.

FIG. 4 illustrates information exchange between the information managing company 300 and a contracted institution 500 which has concluded a contract related to provision of information with the information managing company 300. As described above, it is possible to gather information of the simple diagnostic results aggregated from the diagnostic apparatus 201 installed in a plurality of the diagnostic apparatus installation facilities 200 to clarify an occurrence state of a disease in each group of an area, age, sex, or the like.

The information can be provided to a party other than the user 100 or the contracted medical institution 400. The information managing company 300 provides a password for a contracted institution at the time of concluding a contract with the contracted institution 500. The contracted institution 500 can access the information processing apparatus 301 and input the password to obtain information about an occurrence state of a disease. The contracted institution 500 can rapidly take preventive measures for a target group on the basis of the obtained information.

### REFERENCE SIGNS LIST

1 diagnostic service system

100 user

101 information processing apparatus

200 diagnostic apparatus installation facility

201 diagnostic apparatus

202 disposable device

300 information managing company

301 information processing apparatus

400 contracted medical institution

500 contracted institution

1. A diagnostic service system comprising:
a diagnostic apparatus installed in a diagnostic apparatus installation facility and performing a diagnosis using a disposable device;
a user terminal owned by a user of the diagnostic apparatus; and
an information processing apparatus connected with the diagnostic apparatus and the user terminal and gathering a diagnostic result of the diagnostic apparatus,
wherein the information processing apparatus manages information of the diagnostic result diagnosed by the diagnostic apparatus and information of the user by associating the both with each other, and provides the diagnostic result in accordance with an access from the user terminal of the user.

2. The diagnostic service system according to claim 1,
wherein the disposable device includes a lot number for identifying the disposable device,
the diagnostic apparatus reads the lot number of the disposable device to select a predetermined diagnostic process from a plurality of diagnostic processes stored in advance.

3. The diagnostic service system according to claim 2,
wherein the information processing apparatus causes the user terminal to display an address for accessing the information of the diagnostic result on the user terminal, and provides the information of the diagnostic result and a lot number of the diagnostic result in a case where the address displayed on the user terminal is accessed by the user.

4. The diagnostic service system according to claim 3,
further comprising an information processing apparatus of a medical institution connected to the user terminal or the information processing apparatus,
wherein the information processing apparatus
provides information of the medical institution to the user terminal in accordance with the information of the diagnostic result, and
the information processing apparatus provides information of the user and the information of the diagnostic result which is associated with the information to an information processing terminal of the medical institution in accordance with a request of the user.

5. The diagnostic service system according to claim 4,
wherein the information processing apparatus manages information of a discounted amount of a clinical consultation fee in the medical institution in a case where the user provides the diagnostic result to the medical institution.

6. The diagnostic service system according to claim 5,
wherein the information processing apparatus of the medical institution notifies the information processing apparatus of the lot number of the diagnostic result provided to the user, and
the information processing apparatus provides the information of the diagnostic result of the user which is associated with the notified lot number of the diagnostic result to the information processing apparatus of the medical institution.

7. The diagnostic service system according to claim 6,
wherein the information processing apparatus of the medical institution sends information of a diagnostic result of the user in the medical institution to the information processing apparatus.

8. The diagnostic service system according to claim 1,
wherein the information processing apparatus provides the information of the medical institution selected in accordance with the diagnostic result to the user terminal in a case where the diagnostic result indicates positive, and
the information processing apparatus provides information of a product handled by a diagnostic apparatus installation facility where the diagnostic apparatus is installed to the user terminal in a case where the diagnostic result indicates negative.

9. The diagnostic service system according to claim 1,
wherein the information processing apparatus prepares an electronic medical chart in which a diagnostic result history, diagnosis date, diagnosis place, and personal information of the user are recorded by the diagnostic apparatus for each user.

10. A diagnostic service method in a diagnostic service system including a diagnostic apparatus installed in a diagnostic apparatus installation facility, a user terminal owned by a user of the diagnostic apparatus, an information processing apparatus connected with the diagnostic apparatus and the user terminal and gathering a diagnostic result of the diagnostic apparatus, the diagnostic service method comprising:
a step of, by the diagnostic apparatus, sending the diagnostic result and contact information of the user to the information processing apparatus of an information manager;
a step of, by the information management apparatus, sending an address in which information of the diagnostic result is written;
a step of, by the user terminal, accessing the address in which the diagnostic result is written in accordance with an input by the user to obtain information of the diagnostic result, a lot number of the diagnostic result, and the medical institution;
a step of providing the lot number of the diagnostic result to an information processing terminal of the medical institution through the user terminal in a case where the user has consultation in the medical institution;
a step of, by the information processing terminal of the medical institution, sending the lot number of the diagnostic result to the information processing apparatus to acquire the information of the diagnostic result; and
a step of, by the information processing terminal of the medical institution, sending a diagnostic result of the medical institution to the information processing apparatus.
